(19) United States
(12) Patent Application Publication
(10) Pub. No.: US 2020/0229964 A1
(43) Pub. Date: Jul. 23, 2020

(54) MALE EXTERNAL CATHETER

(71) Applicants: AMINE STAALI, SHARJAH (AE); SOUHEIL GUESSOUM, ALGIERS (DZ)

(72) Inventors: AMINE STAALI, SHARJAH (AE); SOUHEIL GUESSOUM, ALGIERS (DZ)

(21) Appl. No.: 16/747,577

(22) Filed: Jan. 21, 2020

Related U.S. Application Data
(60) Provisional application No. 62/794,805, filed on Jan. 21, 2019.

(51) Int. Cl. A61F 5/453; A61F 5/44

(52) U.S. Cl. CPC A61F 5/453; A61F 5/4408; A61F 5/4407

(57) ABSTRACT

A reusable, non-adhesive, non-invasive male external catheter and a male catheter system for management of urinary incontinence in a male subject. The male catheter system comprises of a male catheter and a support system for said male catheter. The male catheter comprises an isolation chamber having a proximate end, a distal end. The proximate end has an upper proximate body and a lower proximate body, where the upper proximate body of the proximate end of the isolation chamber has a plurality of fixing means; an inlet for genital comprising a retractable non-adhesive sheath to receive and hold the genital while in use; an inlet for fluid; a spraying apparatus comprising a channel for fluid and a plurality of nozzles. The distal end has a drainage outlet comprising a drainage opening.

### FIELD OF THE INVENTION

The present invention relates to a reusable, non-adhesive, non-invasive male external catheter and a male catheter system for management of urinary incontinence in a male subject. More particularly, it relates to a wearable and comfortable urinary external catheter made of a bio-compatible and skin friendly material to fit the contour of male genitalia.

### BACKGROUND OF THE INVENTION

Urinary incontinence is a disease condition that can affect both men and women at any age, but largely affects geriatric patients due to change in functionality of internal organs. The internal organs may behave abnormally due to many reasons like degeneration of tissues, hormonal changes, metabolism and/or any surgical intervention. Urinary incontinence is a specific condition where involuntary muscles of the urinary bladder lose strength and sphincter muscles lose control resulting in continuous leakage of urine from the bladder. It is known in the state of the art that between 15 and 35% of the old age population particularly ages over 60 years old are more prone to this condition. With advancement of the age, the prevalence of disease increases. Male urinary incontinence is becoming increasingly prevalent and its management has become a subject of increased interest for urologists and for other healthcare providers due to the rising elderly population and the risk factors associated as bladder outlet obstruction, neurological disease, cognitive impairment, increase of surgical interventions in cases of prostate cancer, and other urological ailment including but not limited to enuresis, urge incontinence, stress urinary incontinence, functional incontinence amongst others.

While it is not a life-threatening condition, it can significantly impact daily life of an individual affecting mental health and creating an impact on quality of life. The effects of incontinence are many, including severe embarrassment, restriction in daily activities and social life as well as limiting the ability to work and travel. At times, this may also create fear, rage, and shame and sleep disorders due to uncontrollable continuous urinary loss.

US20050101924 A1 discloses male urinary incontinence sheath system including conformable silicone gel and elastic securement tape. A male urinary incontinence device comprises a cylindrical sheath with an open first end to receive the penis and a tubular connector at a second end for discharge of collected urine. A viscous gel strip is applied to the penis of a user and then to the upper inner circumference of the sheath as the sheath is applied to the penis. An elastic strap is applied to the upper outer circumference of the sheath, the gel strip and strap acting cooperatively to retain the sheath on the penis and to render the sheath leak proof in use. The sheath also includes a connector assembly for attachment of tubing and a urine collection vessel.

EP298634 describes an external male catheter. A method of making a tubular elastic sheath (10) having an adhesive-coated inner surface and particularly suitable for use as an external male catheter in a urine-collection system. In such a method, the sheath (10) is supported upon a mandrel (17) and a flexible laminated tape (20), composed of first and second adhesive layers (21 and 22) separated by an elastomeric layer (23), is placed in adhesive contact with the inner surface of the sheath (10), or alternatively, with its outer surface. Thereafter, the cylindrical body of the sheath (10) is rolled upon the mandrel (17) so that the tape (20) is interposed between successive turns of the sheath (10), with the first adhesive layer (21) in engagement with the sheath's inner surface and the second adhesive layer (22) of the tape in contact with the sheath's outer surface. The tape's elastomeric core layer (23) has a greater affinity for the second adhesive layer (22) than for the first adhesive layer (21), and the first adhesive layer (21) has less affinity for the elastomeric layer (23) than for the contiguous surface of the sheath (10), so that unrolling of the sheath (10) during application results in a delaminating of the tape (20) with the first adhesive layer (21) adhering to the sheath's inner surface for securing the sheath (10) to the wearer and with the second adhesive layer (22), along with the elastomeric layer (23), remaining on the sheath's outer surface. There is also disclosed a sheath, suitable for use as a male external catheter.

U.S. Pat. No. 4,759,753 describes an external male catheter having an elastic sheath section and a receiving section connected together when the device is used as an external catheter but constructed to permit detachment of the receiving section, while retaining the sheath section in adhesive attachment to the wearer, to permit direct access to the urethral meatus for intermittent internal catherization. The invention lies in providing an external catheter composed of two main sections—an elastic sheath section and a urine-receiving section that are connected together when the device is in use but may be readily separated when internal catherization is required. Since the two sections are coupled together in close proximity to the urethral meatus, detachment of the urine-receiving section exposes the meatus for internal catheterization. Because only the urine-receiving section needs to be removed, the sheath section and its adhesive attachment to the penile shaft may remain undisturbed.

Therefore, there is a need to provide a concept of penis half elastic sheath inside a urinary bag of male catheter which is adhesive free proven to be good for skin. There is a need of catheter which is non adhesive, remains in a desired position, and has provision of cleaning and drying the penis to obviate infections and other associated discomforts.

### OBJECT OF THE INVENTION

The main object of the present invention is to provide a reusable, non-invasive male external catheter and a male catheter system for management of urinary incontinence in a male subject.

Another object of the present invention is to provide a comfortable and wearable male external catheter made of a bio-compatible and skin friendly material which allows the urine to pass down to the urine collection device without any obstruction.

Yet another object of the present invention is to provide a reusable male external catheter and a male catheter system designed to comfortably fit the contours of male genitalia.

Yet another object of the present invention is to provide a reusable male external catheter designed to be completely discreet while in use.

Yet another object of the present invention is to provide a reusable male external catheter and a male catheter system secured in desired position respective to the body without any adhesives.

Yet another object of the present invention is to provide a reusable male external catheter having a spraying apparatus for hygienically cleaning the genitals and internal walls of the catheter to limit the microbial growth.

Still another object of the present invention is to provide an easy wearable male external catheter and male catheter system for non-urine incontinence patients and ordinary users who have limited toilet options or limited time such as drivers, road travelers, miners, soldiers, security guards, surgeons, pilots, critical site workers, handicapped, special needs and pilgrims to increase their productivity and mobility.

### SUMMARY OF THE INVENTION

The present invention relates to a reusable, non-invasive external catheter and a male catheter system for management of urinary incontinence in a male subject. More particularly, it relates to a wearable and comfortable external catheter made of bio-compatible and skin friendly material such as medical grade silicone, bio-based polymers, copolymers, etc. to fit the contour of male genitalia.

In a preferred embodiment of the present invention is provided a male catheter system comprising a male catheter and a support system for said male catheter. The male catheter comprises an isolation chamber having a proximate end, a distal end. The proximate end has an upper proximate body and a lower proximate body, where the upper proximate body of the proximate end of the isolation chamber has a plurality of fixing means; an inlet for genital comprising a retractable non adhesive sheath to receive and hold the genital while in use; an inlet for fluid; a spraying apparatus comprising a channel for fluid and a plurality of nozzles. The distal end has a drainage outlet comprising a drainage opening. The support system comprises a fore region, an aft region, a top region and a bottom region. The support system holds the catheter in such a manner that it remains at its place around the genitalia allowing the user to engage in various regular activities without getting any fluid leaked out of the catheter. The catheter is highly flexible at proximate end avoiding any discomfort to the user.

In another embodiment of the present invention is provided a support system comprising a supporting wear having a front side and a rear side with a plurality of eyelets for securing the position of catheter; and a wearable attachment; an optional bottom wear having a plurality of eyelets and a flap; said male catheter is attached on wearable attachment at the fore region of said support system through a plurality of attachment means.

In yet another embodiment of the present invention is provided a male catheter comprising an isolation chamber; an inlet for genital comprising a retractable non-adhesive sheath to receive and hold genital while in use; an inlet for fluid; a spraying apparatus comprising a channel for fluid and a plurality of nozzles; and a drainage outlet comprising a drainage opening. The isolation chamber has a proximate end and a distal end, said proximate end further comprises an upper proximate body and a lower proximate body, said upper proximate body of the proximate end of the isolation chamber having a plurality of fixing means, the upper proximate body of said isolation chamber is positioned such that said inlet for genital is surrounded by said isolation chamber. The inlet for fluid is positioned preferably on upper proximate body, said inlet for fluid being connected to the spraying apparatus forming a continued channel to direct a fluid to pass through the inlet towards spraying apparatus and to enter said isolation chamber. The inlet for fluid is optionally connected to a portable bidet and said bidet provides pressurized fluid. The spraying apparatus is positioned, inside and around the proximate end of the isolation chamber. The drainage outlet is positioned at distal end of said isolation chamber such that the drainage opening of said outlet faces isolation chamber, and said drainage opening protrudes allowing fluid collected in isolation chamber to be drained out.

In an additional embodiment of the present invention is provided an external male catheter comprising a holding, an isolation chamber, an inlet for genital, an inlet for fluid, a spraying apparatus and a drainage outlet. The holding has a distal portion, a proximate portion, an upper portion and a lower portion, a scrotum supporting means, a plurality of loop connectors at said upper and said lower portions of said holding. The isolation chamber comprises a proximal end and a distal end. The inlet for genital comprises a retractable, elastic, non-adhesive sheath having a fixed end to receive the genital and a protruding end to hold genital till upper half length of genital while in use. The inlet for genital is positioned at upper-proximate portion of said holding such that said inlet for genital allows genital to be inserted into said inlet for genital; the proximal end of said isolation chamber is connected to the upper-proximate portion of said holding such that said inlet for genital is surrounded by said isolation chamber. The inlet for fluid is positioned preferably towards upper portion of said holding, said inlet for fluid being connected to the spraying apparatus forming a continued channel to direct a fluid to pass through the inlet towards spraying apparatus and to enter into said isolation chamber. The inlet for fluid is optionally connected to a portable bidet and said bidet provides pressurized fluid. The spraying apparatus comprises a channel for fluid and a plurality of nozzles. The spraying apparatus is positioned, inside and around the proximal end of the isolation chamber. The drainage outlet comprises a receiving opening and a drainage opening. Said drainage outlet being positioned at distal-lower portion of said isolation chamber such that the receiving opening of said outlet faces inner isolation chamber, and said drainage opening protrudes out of said holding allowing fluid collected in isolation chamber to be drained out.

In a preferred embodiment of the present invention is provided a male catheter system comprising a male catheter and a support system for the male catheter, wherein, said male catheter comprises an isolation chamber comprising a proximate end, a distal end and said proximate end further comprises an upper proximate body and a lower proximate body, said upper proximate body of the proximate end of the isolation chamber having a plurality of fixing means; an inlet for genital comprising a retractable non adhesive sheath to receive and hold the genital while in use; an inlet for fluid; a spraying apparatus comprising a channel for fluid and a plurality of nozzles; and a drainage outlet comprising a drainage opening; said support system has a fore region, an aft region, a top region and a bottom region.

In another embodiment of the present invention is provided a support system comprising a supporting wear having a front side and a rear side with a plurality of eyelets for securing the position of catheter; and a wearable attachment; an optional bottom wear having a plurality of eyelets and a flap; said male catheter is attached on wearable attachment at the fore region of said support system through a plurality of attachment means.

In yet another embodiment of the present invention is provided a male catheter comprising an isolation chamber comprising a proximate end and a distal end, said proximate end further comprises an upper proximate body and a lower proximate body, said upper proximate body of the proximate end of the isolation chamber having a plurality of fixing means; an inlet for genital comprising a retractable non adhesive sheath to receive and hold genital while in use; an inlet for fluid; a spraying apparatus comprising a channel for fluid and a plurality of nozzles; and a drainage outlet comprising a drainage opening; wherein; the upper proximate body of said isolation chamber is positioned such that said inlet for genital is surrounded by said isolation chamber; the inlet for fluid is positioned preferably on upper proximate body, said inlet for fluid being connected to the spraying apparatus forming a continued channel to direct a fluid to pass through the inlet towards spraying apparatus and to enter into said isolation chamber; the spraying apparatus is positioned, inside and around the proximate end of the isolation chamber; and said drainage outlet being positioned at distal end of said isolation chamber such that the drainage opening of said outlet faces isolation chamber, and said drainage opening protrudes allowing fluid collected in isolation chamber to be drained out; the inlet for fluid is optionally connected to a portable bidet and said bidet provides pressurized fluid.

In an embodiment the male external catheter is connected to an external bidet through the inlet for fluid. The external catheter thus provides a convenience of washing the genital and cleaning the chamber. The bidet includes but is not limited to an automatic bidet, a manual bidet, a fluid container or similar structures known in the art.

### BRIEF DESCRIPTION OF THE DRAWING

An understanding of the device and method of the present invention may be obtained by reference to the following non limiting drawings:

FIG. 1 shows an exploded view of male catheter system in accordance with an embodiment of the present invention.

FIG. 2(a) shows the perspective view of male catheter system in accordance with an embodiment of the present invention.

FIG. 2(b) shows the arrangement of male catheter system in accordance with an embodiment of the present invention.

FIG. 2(c) shows the rear side of the bottom wear of the support system of male catheter system in accordance with an embodiment of the present invention.

FIGS. 3(a) and 3(b) shows an exploded view of isolation chamber of male catheter in accordance with an embodiment of the present invention.

FIGS. 4(a) and 4(b) shows the perspective and front view of supporting wear of male catheter system in accordance with an embodiment of the present invention.

FIG. 5 shows the perspective view of wearable attachment of male catheter system in accordance with an embodiment of the present invention.

FIGS. 6(a) and 6(b) shows the perspective view and sectional view of spraying apparatus in accordance with an embodiment of the present invention.

FIGS. 7(a), 7(b) and 7(c) shows the front view, perspective view of the isolation chamber and sectional view of drainage outlet of male catheter in accordance with an embodiment of the present invention.

FIG. 8(a) is a perspective view of a male catheter and male catheter system in accordance with an embodiment of the present invention.

FIGS. 8(b) and 8(c) show the front view of supporting wear of male catheter in accordance with an embodiment of the present invention.

FIG. 9(a) is an exploded perspective side view of an external male catheter in accordance with an additional embodiment of the present invention.

FIG. 9(b) is an exploded perspective side view of an external male catheter in accordance with additional embodiment of the present invention.

FIG. 10(a) is a rear view of the external male catheter in accordance with an additional embodiment of the present invention.

FIG. 10(b) is a front view of the external male catheter in accordance with an additional embodiment of the present invention.

FIGS. 10(c) and 10(d) depict a front view of the external male catheter in accordance with an additional embodiment of the present invention.

FIG. 11(a) is a front view of spraying apparatus of the male catheter in accordance with an additional embodiment of the present invention.

FIG. 11(b) is a top perspective view of spraying apparatus of the male catheter in accordance with an additional embodiment of the present invention.

FIG. 11(c) is a sectional view of spraying apparatus of the male catheter in accordance with an additional embodiment of the present invention.

FIGS. 11(d) and 11(e) show spraying apparatus of the male catheter in accordance with an additional embodiment of the present invention.

FIG. 12 is a perspective view of retractable, non-adhesive sheath of the male catheter in accordance with an additional embodiment of the present invention.

FIG. 13(a) is a perspective view of isolation chamber of the male catheter in accordance with an additional embodiment of the present invention.

FIG. 13(b) is a perspective view of isolation chamber of the male catheter in accordance with additional embodiment of the present invention.

FIG. 14(a) is a front perspective view of holding of the male catheter in accordance with an embodiment of the present invention.

FIG. 14(b) is a front perspective view of holding of the male catheter in accordance with additional embodiment of the present invention.

### DETAILED DESCRIPTION OF THE INVENTION

Many aspects of the invention can be better understood with references made to the drawings below. The components in the drawings are not necessarily drawn to scale. Instead, emphasis is placed upon clearly illustrating the components of the present invention. Moreover, like reference numerals designate corresponding parts through the several views in the drawings. Before explaining at least one embodiment of the invention, it is to be understood that the embodiments of the invention are not limited in their application to the details of construction and to the arrangement of the components set forth in the following description or illustrated in the drawings. The embodiments of the invention are capable of being practiced and carried out in various ways. In addition, the phraseology and terminology employed herein are for the purpose of description and should not be regarded as limiting.

In a preferred embodiment of the present invention is provided a male catheter system comprising a male catheter and a support system for the male catheter, wherein, said male catheter comprises an isolation chamber comprising a proximate end, a distal end and said proximate end further comprises an upper proximate body and a lower proximate body, said upper proximate body of the proximate end of the isolation chamber having a plurality of fixing means; an inlet for genital comprising a retractable non adhesive sheath to receive and hold the genital while in use; an inlet for fluid; a spraying apparatus comprising a channel for fluid and a plurality of nozzles; and a drainage outlet comprising a drainage opening; said support system comprises a holding having a fore region, an aft region, a top region and a bottom region.

In another embodiment of the present invention is provided a support system 1 comprising a supporting wear having a front side and a rear side with a plurality of eyelets for securing the position of catheter; and a wearable attachment; an optional bottom wear having a plurality of eyelets and a flap; said male catheter is attached on wearable attachment at the fore region of said support system through a plurality of attachment means.

In yet another embodiment of the present invention is provided a male catheter comprising an isolation chamber comprising a proximate end and a distal end, said proximate end further comprises an upper proximate body and a lower proximate body, said upper proximate body of the proximate end of the isolation chamber having a plurality of fixing means; an inlet for genital comprising a retractable non adhesive sheath to receive and hold genital while in use; an inlet for fluid; a spraying apparatus comprising a channel for fluid and a plurality of nozzles; and a drainage outlet comprising a drainage opening; wherein; the upper proximate body of said isolation chamber is positioned such that said inlet for genital is surrounded by said isolation chamber; the inlet for fluid is positioned preferably on upper proximate body, said inlet for fluid being connected to the spraying apparatus forming a continued channel to direct a fluid to pass through the inlet towards spraying apparatus and to enter into said isolation chamber; the spraying apparatus is positioned, inside and around the proximate end of the isolation chamber; and said drainage outlet being positioned at distal end of said isolation chamber such that opening of said drainage outlet faces isolation chamber, and said drainage outlet protrudes allowing fluid collected in isolation chamber to be drained out; the inlet for fluid is optionally connected to a portable bidet and said bidet provides pressurized fluid.

In an additional embodiment of the present invention is provided a male external catheter comprising a holding, an isolation chamber, an inlet for genital, an inlet for fluid, a spraying apparatus and a drainage outlet. The holding comprises a distal portion, a proximate portion, an upper portion and a lower portion, and a scrotum supporting means for securing scrotum in position, a plurality of loop connectors at upper and lower portions of said holding. The proximate portion of the holding has slightly curved plain to adjust to the contour of the male user. The inlet for genital is positioned at upper-proximate portion of said holding such that said inlet for genital allows genital to be inserted into said inlet. The inlet for genital comprises a retractable, non-adhesive sheath having a fixed end to receive the genital and a protruding end to hold the genital till upper half length of the genital while in use. The isolation chamber comprises a proximal end and a distal end. The proximal end of said isolation chamber is connected to the upper-proximate portion of said holding such that said inlet for genital is surrounded by said isolation chamber. The proximal end portion of isolation chamber has a flexible body, while the distal end portion of the isolation chamber has a rigid body. More particularly, the flexibility decreases while moving downwards the isolation chamber, such that the proximate end is highly flexible, middle section being moderately flexible and the distal end being rigid. This helps the catheter to function non-disruptively while the user is wearing layers of cloths adding force on isolation chamber. The inlet for fluid is positioned preferably towards upper portion of said holding, said inlet for fluid being connected to the spraying apparatus forming a continued channel to direct a fluid to pass through the inlet towards spraying apparatus and to enter into said isolation chamber. The fluid may be water, air or douche. The spraying apparatus is positioned, inside and around the proximal end of the isolation chamber. The drainage outlet comprises a receiving opening and a drainage opening, said drainage outlet being positioned at distal-lower portion of said isolation chamber such that the receiving opening of said outlet faces inner isolation chamber, and said drainage opening protrudes out of said holding allowing fluid collected in isolation chamber to be drained out. The scrotum supporting means secures scrotum in comfortable position. The scrotum supporting means comprises of two open flexible straps with holders. The straps are made of bio-compatible material such as medical grade silicone, bio-based polymers, copolymers, etc. Alternately, said scrotum supporting means may be in the form of a scrotum ring of a bio-compatible material like silicone, bio-based polymers, copolymers, etc.

In another embodiment the male external catheter is connected to an external bidet through the inlet for fluid. The external catheter thus provides a convenience of washing the genital and cleaning the chamber. The bidet includes but is not limited to an automatic bidet, a manual bidet, a fluid container or similar structures known in the art.

In yet another embodiment, the present invention provides a reusable external male catheter having an isolation chamber. The isolation chamber has an internal space enough to allow the body fluid like urine to come inside it while minimizing the body fluid/urine to come into the contact of penis, while completely disallowing it to come into the contact of scrotum, thus avoiding the microbial infection. The body fluid/urine enters the isolation chamber and gets drained into a fluid/urine collection device though a drainage outlet.

In yet another embodiment, the present invention provides an external male catheter having an isolation chamber, a rigid/semi-rigid spraying apparatus, said spraying apparatus having a plurality of nozzles. The nozzles are either present randomly, or in a single row, or in multiple rows spread on surface of the spraying apparatus. The nozzles are preferably positioned at outward and inward periphery of spraying apparatus, respective to its center to give a shower effect. A bidet having douche or water or air is optionally connected to the inlet for fluid for cleaning and drying the genital, elastic sheath and inner walls of chambers. The outwardly positioned nozzles throw water at an angle ranging from 90 to 45 degrees with respect to wall of spraying apparatus and inwardly positioned nozzles throw water straight to the genital hence maximizing the coverage for cleaning the genital, sheath, and inner walls of chamber. The spraying apparatus has ring like geometry including but not limited to circular, polygonal, semi-circular, semi-polygonal shape.

In yet another embodiment of the present invention, the inlet tube has a water proof vent for air to come in, said vent restricts the water and odor to come out from the isolation chamber and also has a bound cover plugged into the inlet tube to make sure that odor does not come out when the bidet is not connected to it.

In yet another embodiment of the present invention, the distal end of the drainage outlet of the catheter is connected to a fluid collection means that includes but is not limited to a fluid bag, a disposable bag, a multi-chambered fluid receiving/holding bag or similar means know in the art.

In yet another embodiment of the present invention, the external catheter further comprises utility means like sensors for sensing plurality of physio-chemical parameters like frequency and amount of urine passed, sensors for sensing biological parameters like presence of blood cells or pus or any other bio-indicators.

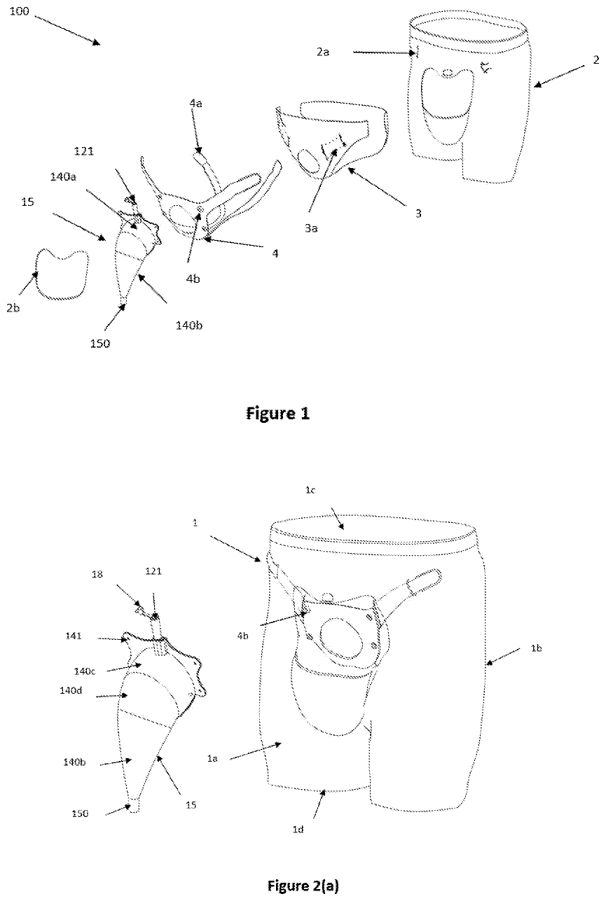

Referring to FIG. 1, an exploded view of the present invention provides the male catheter 100 and male catheter system. The male catheter system comprises a catheter 100 and a support system 1. The male catheter 100 comprises of an isolation chamber 15, an inlet 10 for genital, an inlet 151 for fluid, a spraying apparatus 161 and a drainage outlet 150. The support system 1 has a fore region 1a, an aft region 1b, a top region and a bottom region. Said support system 1 comprises of a bottom wear 2 with a flap 2b on the front region and a plurality of eyelets 2a on front as well as rear region and a supporting wear 3 having a front side 3c and a rear side 3d with a plurality of eyelets 3a for securing the position of genital, a wearable attachment 4 made of a semi-rigid body with a plurality of straps 4a and a plurality of attachment means 4b. The supporting wear 3 is secured to the bottom wear 2 through a securing means. At least two straps 4a of the wearable attachment 4 are inserted through the plurality of eyelets 3a on the front side of the supporting wear 3 and subsequently through the eyelets 2a on the front portion of the bottom wear 2 and at least two straps 4a are taken out from the rear portion of the bottom wear 2 through the rear side of the supporting wear 3 and secured to the rear side of said bottom wear 2 by various joining means. The straps 4a penetrate from the fore region 1a of the support system 1 and exit from the rear region 1b of the support system 1. The inlet 121 for fluid is positioned towards the upper region of said support system 1. Said inlet 121 for fluid is connected to the spraying apparatus 161 forming a continued channel to direct a fluid to pass through the inlet 121 towards spraying apparatus 161 to enter said isolation chamber 15. The fluid includes but is not limited to water, air and douche. The isolation chamber 15 comprises a proximate end 140a and a distal end 140b. The proximate end 140a further comprises of an upper proximate body 140c and a lower proximate body 140d. The upper proximate body 140c of the proximate end 140a of said isolation chamber 15 has a plurality of fixing means 141 and is connected to the fore region 1a of said support system 1 such that said inlet 10 for genital is surrounded by said isolation chamber 15. The upper proximate body 140c of isolation chamber 15 is a soft flexible body, the lower proximate body 140d is a semi-rigid body and the distal end 140b of isolation chamber 15 is a rigid body. This helps the catheter to function non-disruptively while user is wearing layer of clothes adding force on isolation chamber 15. The distal end 140b of said isolation chamber 15 has a drainage outlet 150 comprising a drainage opening 132 and said drainage outlet 150 allows fluid collected in isolation chamber 15 to be drained out. The fixing means 141 on the upper proximate body 140c of the proximate end 140a of the isolation chamber 15 is affixed to the attachment means 4b of the wearable attachment 4 and said wearable attachment 4 is attached to the supporting wear 3 and the bottom wear 2 through the plurality of straps 4a thus completing the arrangement of the male catheter 100. The bottom wear 2 comprises of a plurality of eyelets 2a and a flap 2b act as covering of the support system. The fixing means 141 and attachment means 4b include but are not limited to buttons with eyelets, hooks, snap fasteners, press buttons. The flap 2b is fixed to the bottom wear 2 either through stitching or through a fastening means. The fastening means include but is not limited to buttons with eyelets. The proximal end 140a of isolation chamber 15 has a flexible body, while the distal end 140b of the isolation chamber 15 has a rigid body. More particularly, the flexibility decreases while moving downwards the isolation chamber 15, such that the proximate end is highly flexible, middle section being moderately flexible and the distal end being rigid. This helps the catheter to function non-disruptively while the user is wearing layers of cloths adding force on isolation chamber.

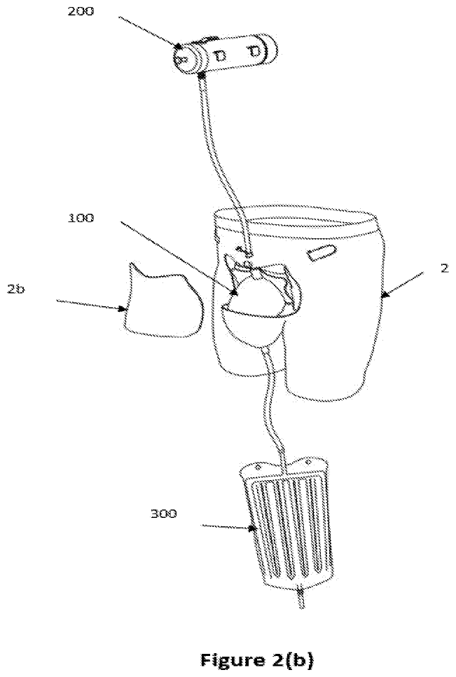

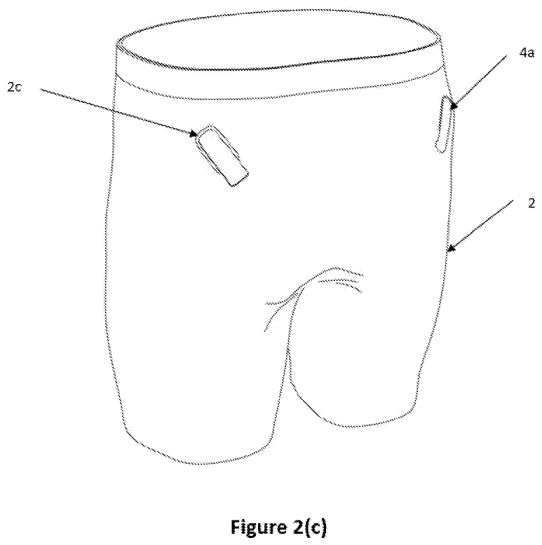

Now referring to FIG. 2(a), the present invention provides the perspective view of male catheter system. The catheter system comprises of a catheter 100 and a support system 1 with its fore region 1a, aft region 1b, upper region 1c, a lower region 1d, an isolation chamber 15. The isolation chamber 15 has a proximate end and a distal end 140b with a plurality of fixing means 141, an inlet 121 for fluid and a drainage outlet 150. The proximate end further comprises of an upper proximate body 140c and a lower proximate body 140d. The inlet 121 for fluid is positioned at the upper region of the support system 1 and is attached to a bidet [not shown] for spraying the genital.

Catheter is attached to the support system 1 through the fixing means 141 and attachment means 4b. The fixing means 141 and attachment means 4b include but not limited to button with eyelets, hook. The proximal end portion of isolation chamber has a flexible body, while the distal end portion of the isolation chamber has a rigid body. More particularly, the flexibility decreases while moving downwards the isolation chamber, such that the proximate end is highly flexible, middle section being moderately flexible and the distal end being rigid. This helps the catheter to function non-disruptively while the user is wearing layers of cloths adding force on isolation chamber. Now referring to FIG. 2(b), the present invention shows the arrangement of male catheter system. The male catheter system comprising male catheter 100 and a support system. The male catheter 100 having an inlet 121 for fluid is attached to the bidet 200 for spraying the genital and the drainage outlet 150 of said male catheter 100 is attached to urine collection device 300 for draining the urine out of the isolation chamber.

Now referring to FIG. 2(c), the present invention shows the rear side of the bottom wear 2. The bottom wear 2 has a plurality of attaching means 2c to attach the plurality of straps 4a of the wearable attachment to said bottom wear 2. The attaching means 2c include but is not limited to Velcro.

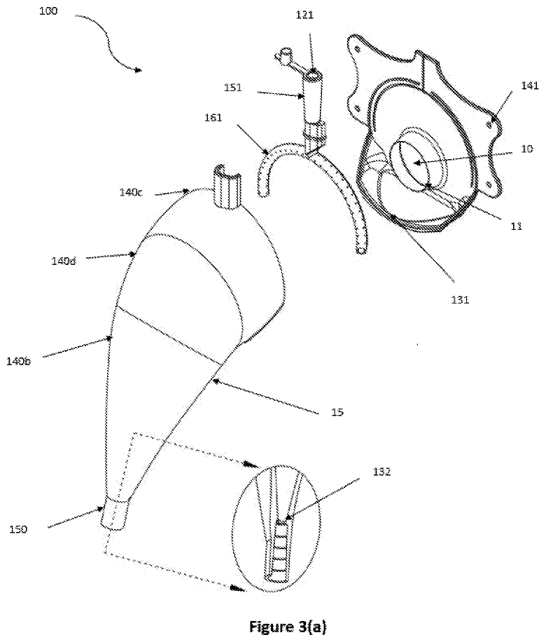

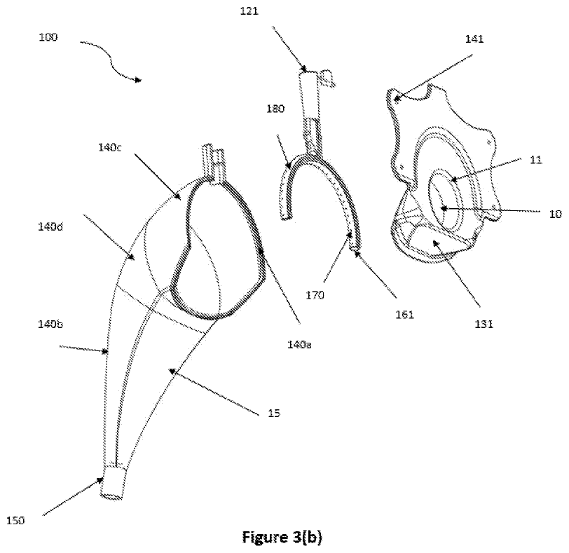

Now referring to FIG. 3(a), the present invention provides an exploded view of male catheter 100. The catheter 100 comprises of an isolation chamber 15, a spraying apparatus 161, a drainage outlet 150, an inlet 121 for fluid, and an inlet 10 for genital. The inlet 10 for genital comprises a non-adhesive sheath 11 to receive the genital and to hold the genital while in use. The isolation chamber 15 comprises a proximate end and a distal end 140b. The proximate end further comprises of an upper proximate body 140c and a lower proximate body 140d. The upper proximate body 140c of the proximate end of said isolation chamber 15 has a plurality of fixing means 141 and is connected to the fore region of said support system such that said inlet 10 for genital is surrounded by said isolation chamber 15. A scrotum supporting wall 131 is positioned below the inlet 10 for genital for providing a comfortable zone that does not put any pressure on the scrotum while tying up the catheter 100 .The upper proximate body 140c of isolation chamber 15 is a soft flexible body, the lower proximate body 140d is a semi-rigid body and the distal end 140b of isolation chamber 15 is a rigid body. This helps the catheter 100 to function non-disruptively while user is wearing layer of clothes adding force on isolation chamber 15. The inlet 121 for fluid is positioned towards the upper region of said support system. Said inlet 121 for fluid is connected to the spraying apparatus 161 forming a continued channel to direct a fluid to pass through the inlet 121 towards spraying apparatus 161 to enter said isolation chamber 15. The fluid includes but is not limited to water, air and douche. The spraying apparatus 161 is positioned inside and around the proximate end of the isolation chamber 15. The distal end 140b of said isolation chamber 15 has a drainage outlet 150 comprising a drainage opening 132 and said drainage outlet 150 allows fluid collected in isolation chamber 15 to be drained out. The fixing means 141 on the upper proximate body 140c of the proximate end of the isolation chamber 15 is affixed to the attachment means of the wearable attachment and said wearable attachment is attached to the supporting wear and the bottom wear through the plurality of straps thus completing the arrangement of the male catheter 100. The fixing means 141 and attachment means including but is not limited to buttons with eyelets, hooks, snap fasteners, press buttons, loop buttons, Velcro.

Now referring to FIG. 3(b), a perspective view of the present invention provides an exploded view of male catheter 100. The catheter 100 comprises of an inlet 10 for genital, an inlet 121 for fluid, an isolation chamber 15, a spraying apparatus 161, a drainage outlet 150 and a scrotum supporting wall 131 positioned below the inlet 10 for genital for providing a comfortable zone that does not put any pressure on the scrotum while tying up the catheter 100. The isolation chamber 15 has a proximate end 140a and a distal end 140b. The proximate end 140a further comprises of an upper proximate body 140c and a lower proximate body 140d. The inlet 10 for genital comprises of a non-adhesive sheath 11 to hold the genital while in use. The spraying apparatus 161 is provided with outwardly positioned nozzles 180 and inwardly positioned nozzles 170 to make a shower effect through an inlet 121 for fluid.

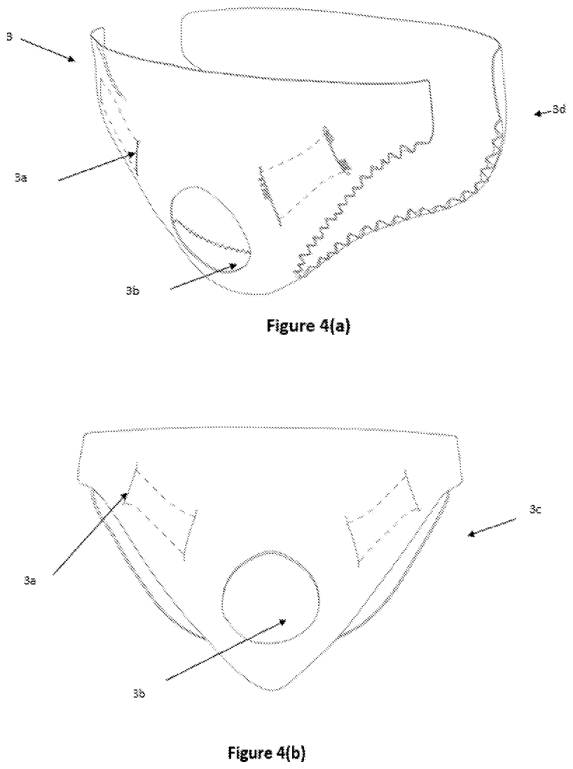

Now referring to FIGS. 4(a) and 4(b), the front side 3c and the back side 3d of supporting wear 3 is shown respectively. The supporting wear 3 has a plurality of eyelets 3a on its front side and back side for inserting the plurality of straps from the fore region of the support system 1 to the aft region of the support system. The supporting wear has an inlet for genital 3b to insert the genital from the fore region through the aft region of the support system. The leg openings of the supporting wear are sewed with elastic bands to conform with the leg of user.

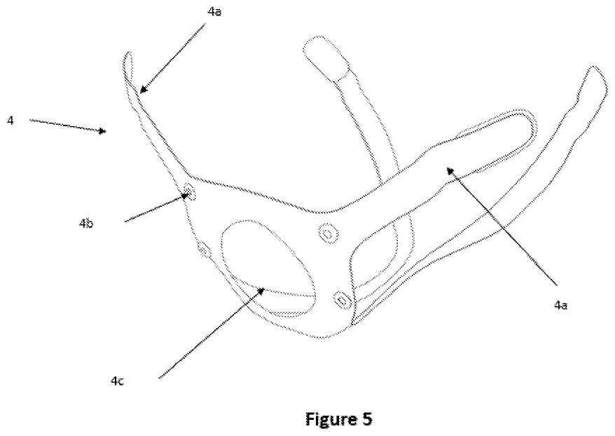

FIG. 5, the perspective view of the present invention is provided depicting wearable attachment 4. The wearable attachment 4 has a plurality of attachment means 4b with a plurality of straps 4a and an inlet 4c for genital. The straps 4a are inserted into the eyelets of the bottom wear through the eyelets of the supporting wear and thus completing the arrangement of the support system. The plurality of straps 4a of the wearable attachment 4 are inserted through the plurality of eyelets on the front side of the supporting wear and subsequently through the eyelets on the front region of the bottom wear and secured on the front region of the bottom wear and said plurality of straps 4a traverse through the rear side of the bottom wear through the rear side of the supporting wear and secured to the rear side of said bottom wear by a joining means. The plurality of straps 4a penetrates from the fore region of the support system and exits from the aft region of the support system.

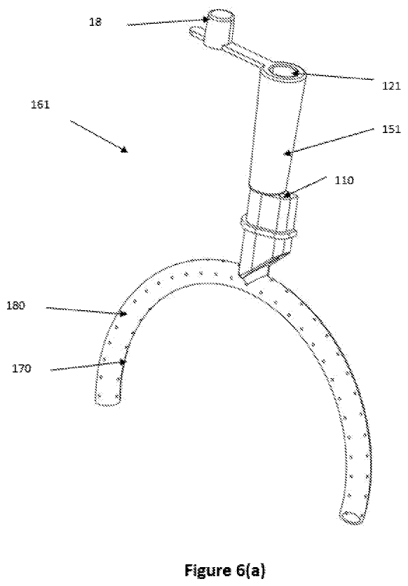

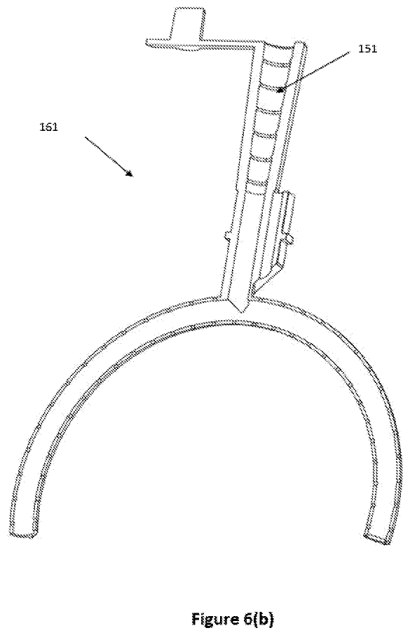

Now referring to FIGS. 6(a) and 6(b), the present invention is depicted through the perspective and sectional view of the spraying apparatus 161 respectively. The spraying apparatus 161 has an inlet 121 for fluid with an inlet tube 151 thus forming a continued channel, a cap 18 to close the inlet 121 when not in use, an air vent 110 to expel the air and odor from the isolation chamber. The spraying apparatus 161 has a plurality of outward positioned nozzles 180 and inward positioned nozzles 170 to make a shower effect through the inlet 121. The inlet 121 is attached to the bidet for spraying the genital. The inlet tube 151 is made up of chambers that lock the inlet pipe of the bidet.

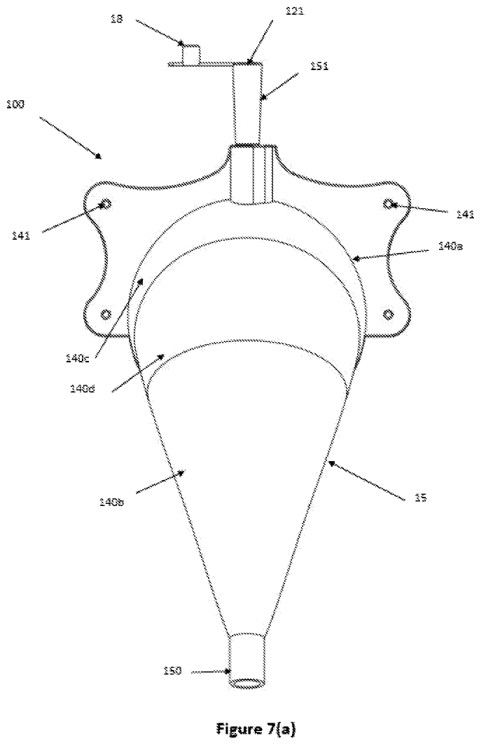

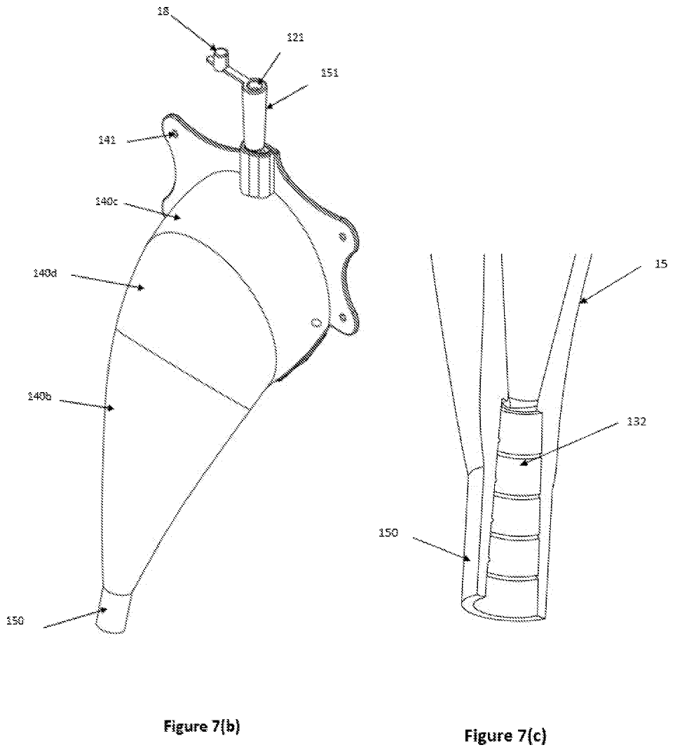

Now referring to FIGS. 7(a) and 7(b), the present invention is depicted by the front view and the perspective view of isolation chamber 15 respectively. The isolation chamber 15 comprises of a proximate end 140a, a distal end 140b, an inlet 121 for fluid with an inlet tube 151 and a cap 18, a plurality of fixing means 141 and a drainage outlet 150. The proximate end 140a further comprises of an upper proximate body 140c and a lower proximate body 140d. The isolation chamber 15 surrounds the genital. The isolation chamber 15 gets attached to the support system through the plurality of fixing means 141. The upper proximate body 140c is a soft material body following the lower proximate body 140d made of a semi-rigid body and finally the distal end 140b is a rigid body thus allowing urine not to come into contact of genital thus preventing bacterial infection. The cap 18 closes the inlet 121 of the fluid when not in use. The drainage outlet 150 is attached to a urine collection device for draining the urine out from the isolation chamber 15.

Now referring to FIG. 7(c), the drainage outlet 150 of the isolation chamber 15 is shown according to the present invention. The drainage outlet 150 has a drainage opening 132 for attaching the drainage outlet 150 to the isolation chamber 15.

In an aspect of the present invention, a method of managing urine collection in a male subject is also provided. The method comprises steps of:
a) wearing an external male catheter, said catheter comprising a holding, an isolation chamber, an inlet for genital comprising a retractable, elastic, non-adhesive sheath, an inlet for fluid, a spraying apparatus comprising a channel for fluid and a plurality of nozzles and a drainage outlet comprising a drainage opening;
b) the catheter is worn by putting male genital in the inlet for genital followed by inserting the genital into the isolation chamber till a comfort fit is achieved while the sheath covers and holds the genital;
c) securing the position of the holding by connecting said plurality of eyelets to either a plurality of external straps or a plurality of ropes;
d) collecting the urine in isolation chamber, washing the urine through the inlet for fluid and spraying apparatus;
e) draining the urine and wash water out through drainage outlet; and
f) drying the genital through the inlet for fluid and spraying apparatus.

The catheter is optionally connected to an external portable bidet through the inlet for fluid. The proximal end portion of isolation chamber has a flexible body, while the distal end portion of the isolation chamber has a rigid body. More particularly, the flexibility decreases while moving downwards the isolation chamber, such that the proximate end is highly flexible, middle section being moderately flexible and the distal end being rigid. This helps the catheter to function non-disruptively while the user is wearing layers of cloths adding force on isolation chamber.

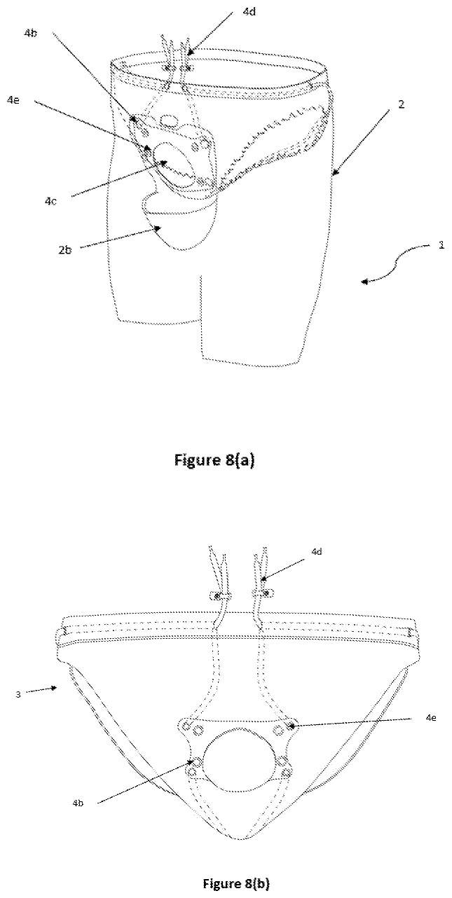

FIG. 8(a)-(c) depict the perspective view of support system 1 with its fore region, aft region, upper region, a lower region, and a flap 2b. The catheter is attached to the support system 1 through the fixing means on catheter and attachment means 4b on support system 1. The fixing means and attachment means include but are not limited to button with eyelets, hook. The wearable attachment 4 with an inlet 4c for genital has a plurality of attachment means 4b with a plurality of ropes 4d and a plurality of holes 4e for channeling the plurality of ropes 4d. The plurality of ropes are looped around the wearable attachment 4 through the holes 4e and run around each thigh of user and come out from the plurality of eyelets 2a [not visible] on the rear side of the bottom wear 2 through the plurality of eyelets 3a [not shown] on the rear side 3d [not visible] of the supporting wear 3. In a preferred embodiment the plurality of holes 4e overlap the plurality of attachment means 4b as shown in FIG. 8(c).

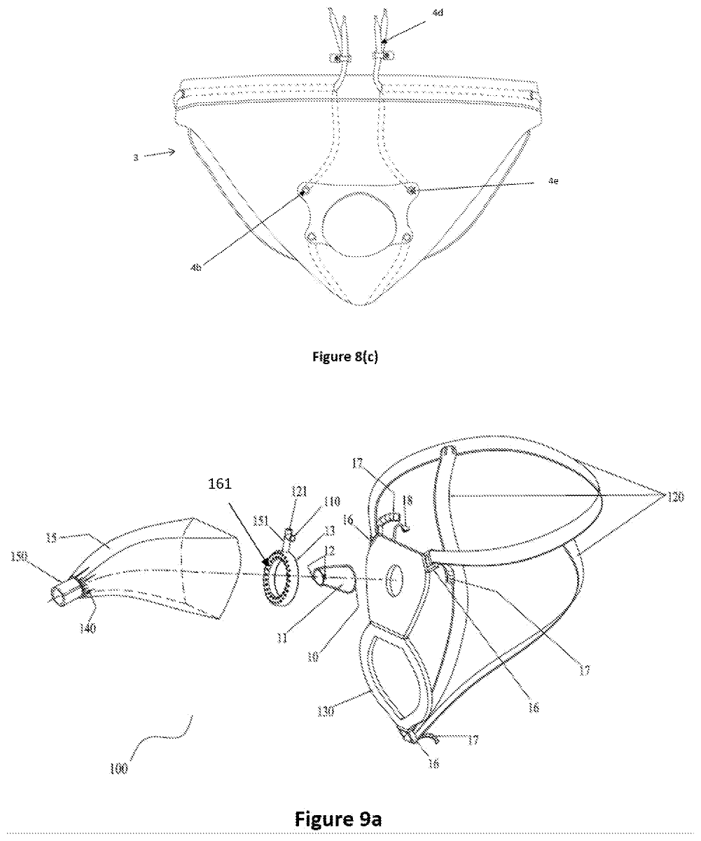

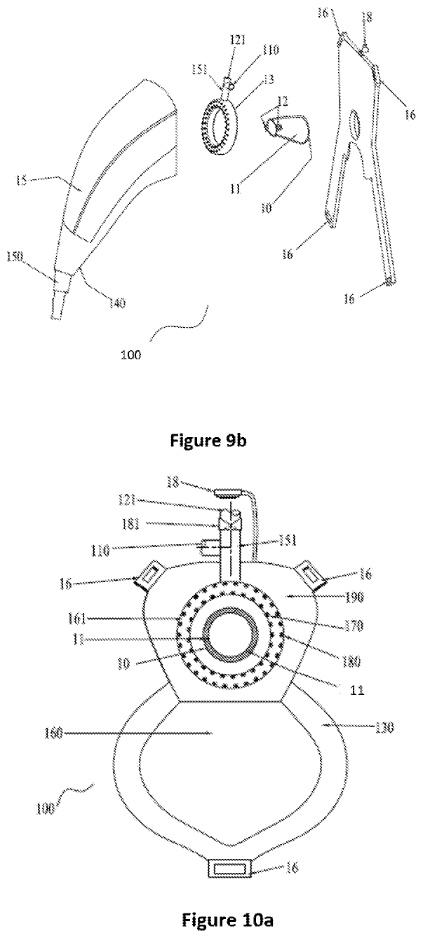

FIG. 9(a) shows an alternate embodiment of the present invention. A side view of reusable male catheter 100 is provided with a retractable sheath 11 having a fixed end 10 and a protruding end 13, said protruding end having sheath handle 12. The sheath receives and holds the genital and is 100-500% elastic to conform the different sizes of penis. The sheath handle 12 is provided to hold the sheath 11. The body fluid/urine enters the isolation chamber 15. The isolation chamber 15 is flexible in nature and has enough space thus not allowing the genital to be in contact with bodily fluid/urine thereby avoiding the microbial infection. The external catheter further comprises a rigid/semi-rigid spraying apparatus 161 having a plurality of nozzles. The spraying apparatus 161 makes a shower effect to clean and dry the genital, sheath, inner walls and surroundings of the isolation chamber 15. The inlet tube 151 is connected to bidet (not shown) having douche or water or air to clean and dry the genitals thus limiting the bacterial growth. The inlet tube 151 has a waterproof vent 110 for air to come in and restricts the water and odor to come out from the isolation chamber 15. The bound cover/cap 18 is plugged in the connector of inlet tube 151 to make sure that the odor does not come out when bidet is not connected. A fitting strap 17 attached to the loop 16 provides a tightening mechanism to get the external catheter well attached to the body. The external catheter further comprises of scrotum supporting means 130 surrounding the scrotum made up of skin friendly and elastic material such as biomedical silicon, bio-based polymers, copolymers, etc. and a loop 16 for correct positioning of genital so to get well attached to the genital. The whole external catheter assembly is comfortable and wearable with an underwear strap 120 which allows the urine to pass down to the urine collection device without any obstructed flow.

The external male catheter may further comprises a half cut condom made of an elastic sheath that surrounds the genital, said sheath having elasticity in range of 100-500% to accommodate different sizes of genital, capable of securing the position respective to the genital without any adhesives.

FIG. 9(b) depicts an exploded perspective side view of an external male catheter in accordance with another embodiment of the present invention. A side view of reusable male catheter 100 is provided with a retractable sheath 11. The isolation chamber 15 is flexible in nature and has enough space thus not allowing the genital to be in contact with bodily fluid/urine thereby avoiding the bacterial infection. More particularly, the proximal end portion of isolation chamber has a flexible body, while the distal end portion of the isolation chamber has a semi-rigid body. This helps the catheter to function non-disruptively while the user is wearing layers of cloths adding force on isolation chamber. A fitting strap attached to the loop 16 provides a tightening mechanism to get the external catheter well attached to the body. The external catheter further comprises of scrotum supporting means in the form of lateral straps and a curvature at lower portion of holding unit thus surrounding the scrotum. The scrotum supporting means is made up of skin friendly and elastic material such as biomedical silicone, bio-based polymers, copolymers, etc. and a loop 16 for correct positioning of genital so to get well attached to the genital. The whole external catheter assembly is comfortable and wearable with an underwear strap which allows the urine to pass down to the urine collection device without any obstructed flow.

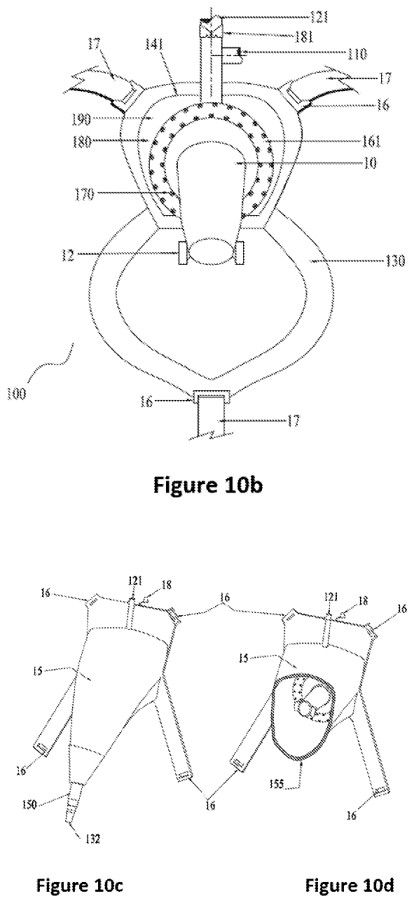

Now referring to FIG. 10(a), in an embodiment of the present invention, a back view of the external catheter 100 is provided with a sheath 11 having a fixed end 10 and a protruding end, said protruding end having sheath handle. The sheath 11 receives the genital from fixed end 10 and hold the genital and is 100-500% elastic to conform to different sizes of penis. It has a rigid/semi-rigid spraying apparatus 161 having a plurality of nozzles. The spraying apparatus 161 makes a shower effect to clean and dry the genital. The inlet tube151 is connected from the top of a bidet (not shown) having douche or water or air for cleaning and drying the genitals, elastic sheath and inner walls of chambers thus limiting the bacterial growth. The outwardly positioned nozzles 180 throw water at an angle ranging from 90 to 45 degrees with respect to wall of spraying apparatus and inwardly positioned nozzles 170 throw water straight to the genital hence maximizing the coverage for cleaning the genital, sheath, and inner walls of chamber. The inlet tube 151 has a water proof vent 110 for air to come in and restricts the water and odor to come out from the isolation chamber and also has a bound cover/cap 18 plugged into the tube 151 connected through a connector 181 to make sure that odor does not come out when bidet is not connected. The catheter further comprises of scrotum supporting means 130, surrounding the scrotum made up of skin friendly and elastic material such as biomedical silicone bio-based polymers, copolymers, etc. and a loop or holder 16 for correct positioning of genital so to get well attached to the genital.

FIG. 10(b), in an embodiment of the present invention, a front view of external catheter is provided with a sheath having a fixed end 10 and a protruding end, said protruding end having sheath handle 12. The sheath receives the genital from fixed end 10 and hold the genital and is 100-500% elastic to conform the different sizes of penis. It has a rigid/semi-rigid spraying apparatus 161 with a plurality of nozzles to make a shower effect to clean and dry the genital. The inlet 121 is connected from the top of a bidet (not shown) having douche or water or air for cleaning and drying the genitals, elastic sheath and inner walls of chambers. The outwardly positioned nozzles 180 throw water at an angle ranging from 90 to 45 degrees with respect to wall of spraying apparatus and inwardly positioned nozzles 170 throw water straight to the genital hence maximizing the coverage for cleaning the genital, sheath, and inner walls of chamber. The inlet 121 has a water proof vent 110 for air to come in and restricts the fluid and odor to come out from the isolation chamber, a bound cover/cap plugged into the inlet tube to make sure that odor does not come out. The external catheter further comprises of scrotum supporting means 130 surrounding the scrotum made up of skin friendly and elastic material such as biomedical silicone, bio-based polymers, copolymers, etc. and a loop 16 for correct positioning of genital so to get well attached to the genital.

FIG. 10(c) depicts a front view of external catheter according to an alternate embodiment of the present invention. FIG. 10(d) depicts a front sectional view of external catheter with partial internal view of isolation chamber. The proximal end portion of isolation chamber has a flexible body, while the distal end portion of the isolation chamber has a semi-rigid body. This helps the catheter to function non-disruptively while the user is wearing layers of cloths adding force on isolation chamber. A fitting strap attached to the loop 16 provides a tightening mechanism to get the external catheter well attached to the body. The external catheter further comprises of scrotum supporting means in the form of lateral straps and a curvature at lower portion of holding unit thus surrounding the scrotum. The scrotum supporting means is made up of skin friendly and elastic material such as biomedical silicone, bio-based polymers, copolymers, etc. and a loop 16 for correct positioning of genital so to get well attached to the genital. The whole external catheter assembly is comfortable and wearable with an underwear strap which allows the urine to pass down to the urine collection device without any obstructed flow. The proximal end portion of isolation chamber has a flexible body, while the distal end portion of the isolation chamber has a rigid body. More particularly, the flexibility decreases while moving downwards the isolation chamber, such that the proximate end is highly flexible, middle section being moderately flexible and the distal end being rigid. This helps the catheter to function non-disruptively while the user is wearing layers of cloths adding force on isolation chamber.

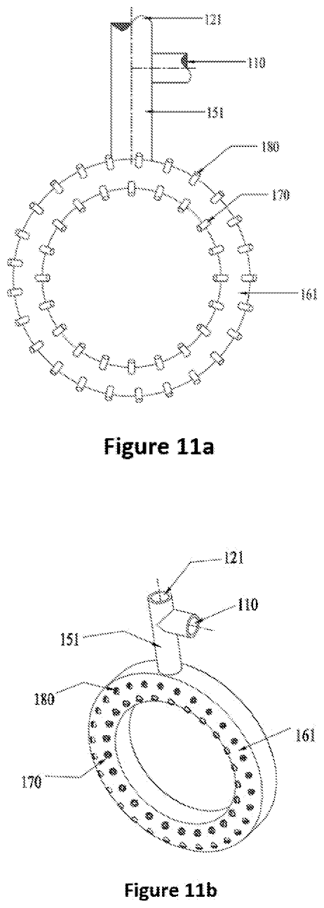

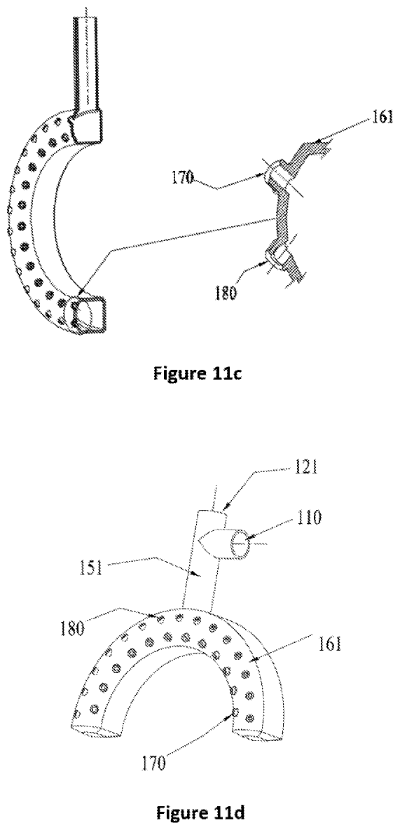

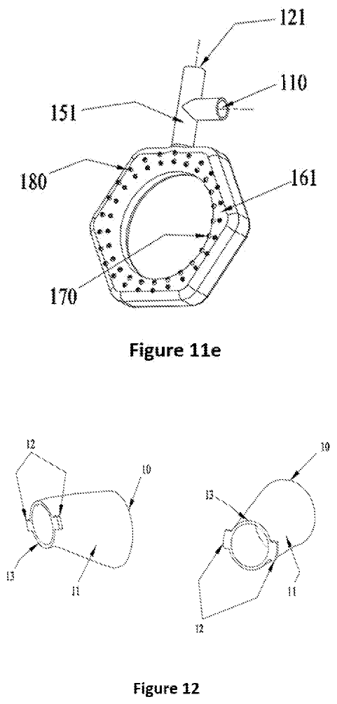

Now referring to FIGS. 11(a), 11(b) and 11(c), in an embodiment of the present invention, a front view of the spraying apparatus 161 is provided with outwardly positioned nozzles 180 and inwardly positioned nozzles 170 to make a shower effect with an inlet tube 151 connected from the top of a bidet (not shown) having douche or water or air for cleaning the genitals, elastic sheath and inner walls of chambers. The outwardly positioned nozzles 180 throw water at an angle ranging from 90 to 45 degrees with respect to wall of spraying apparatus and inwardly positioned nozzles 170 throw water straight to the genital hence maximizing the coverage for cleaning the genital, sheath, and inner walls of chamber. The inlet tube 151 has a water proof vent 110 for air to come in and restricts the water and odor to come out from the isolation chamber.

Now referring to FIG. 11(d), in an alternate embodiment of the present invention, a front view of the spraying apparatus 161 has a semi-circular configuration and is provided with outwardly positioned nozzles 180 and inwardly positioned nozzles 170 to make a shower effect with an inlet tube 151 connected from the top of a bidet (not shown) having douche or water or air for cleaning the genitals, elastic sheath and inner walls of chambers. A different configuration of spraying apparatus is shown in FIG. 11(e) where a polygonal spraying apparatus 161 is provided with outwardly positioned nozzles 180 and inwardly positioned nozzles 170 to make a shower effect with an inlet tube 151.

Now referring to FIG. 12, in an embodiment of the present invention, different views of retractable, non-adhesive sheath 11 having fixed end 10 and protruding end 13 with sheath handles 12.

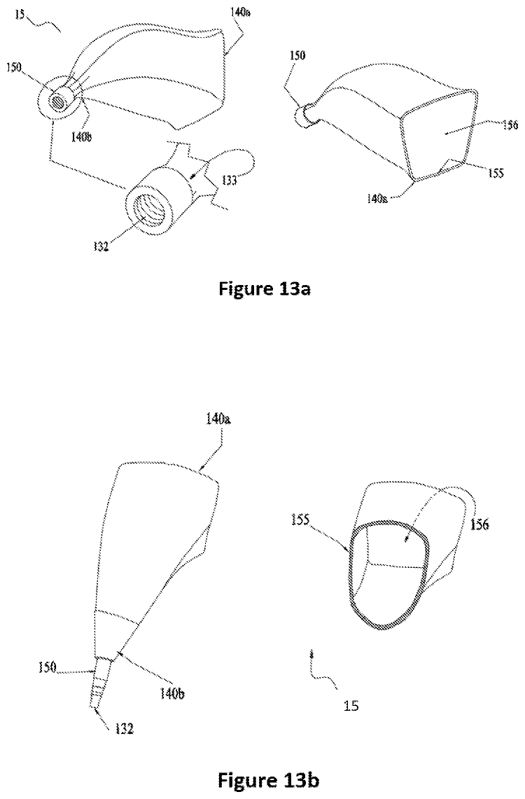

Now referring to FIG. 13(a), in an embodiment of the present invention, different views of isolation chamber 15 show a distal end 140b and proximal end 140a and a drainage outlet 150 connected to said distal end 140b, said drainage outlet 150 comprising a receiving opening 133 and a drainage opening 132. The receiving opening 133 faces internal space 156 and drainage opening protrudes out of distal end 140b.

Now referring to FIG. 13(b), in an embodiment of the present invention, different views of isolation chamber 15 shows a rigid distal end 140b and a flexible proximal end 140a and a drainage outlet 150 connected to said distal end 140b. The proximal end portion of isolation chamber has a flexible body, while the distal end portion of the isolation chamber has a rigid body. More particularly, the flexibility decreases while moving downwards the isolation chamber, such that the proximate end is highly flexible, middle section being moderately flexible and the distal end being rigid. This helps the catheter to function non-disruptively while the user is wearing layers of cloths adding force on isolation chamber.

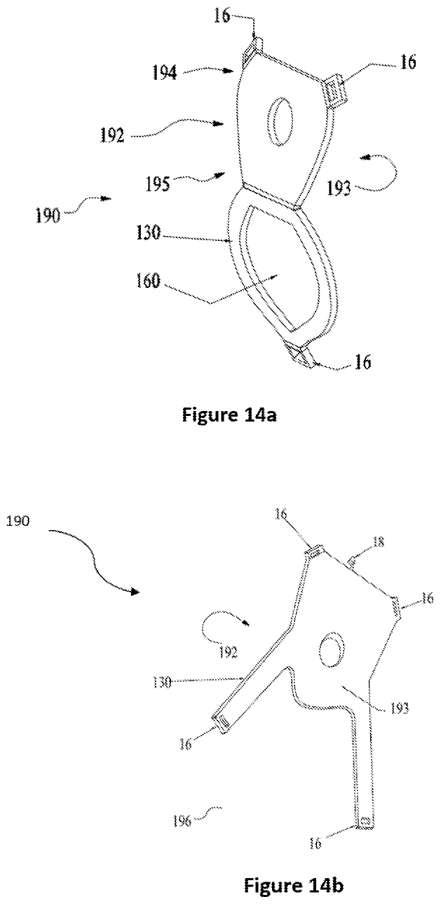

Now referring to FIG. 14(a), in an embodiment of the present invention, a front perspective view of holding 190 having a distal portion 192, a proximate portion 193, an upper portion 194 and a lower portion 195, and a scrotum support means 130 for securing scrotum in position, a plurality of loop connectors 16 at upper and lower portions of said holding 190.

Now referring to FIG. 14(b), in an embodiment of the present invention, a front perspective view of holding 190 having a distal portion 192, a proximate portion 193, an upper portion 194 and a lower portion 195, and a scrotum support means 130 for securing scrotum in position, a plurality of loop connectors 16 at upper and lower portions of said holding. The holding means further comprises of scrotum supporting means 130 in the form of lateral straps and a curvature at lower portion of holding unit thus surrounding the scrotum. The scrotum supporting means is made up of skin friendly and elastic material such as biomedical silicone, bio-based polymers, copolymers, etc. and a loop or holder 16 for correct positioning of genital so to get well attached to the genital.

While wearing, the device includes pulling the sheath out of isolation chamber such that the sheath is inverted, the distal end is opened by pulling apart the handles, and putting the genital in said inverted sheath and pushing the genital along with the sheath in the isolation chamber while forming the sheath a half sleeve like structure on genital.

The catheter in accordance with the present invention provides significant advantages over those known in the art. The catheter of present invention prevents and obviates concerns like bad odor and other associated disadvantages of diapers including but not limited to skin rashes, infection, wet feeling etc. The catheter of present invention is so designed to provide as underwear like feeling and comfort to a user. An important aspect of the present catheter is its utility to provide and easy catheterization option for hospital admitted non-urine incontinence patients which is easy for patients and less costly for hospitals.

List of reference numerals

```
  1: Support system
  1(a): Fore region of support system
  1(b): Aft region of support system
  1(c): Upper region of support system
  1d: Lower region of support system
  2: Bottom wear
  2a: Eyelets in bottom wear
  2b: Flap in bottom wear
  2c: Attaching means in bottom wear
  3: Supporting wear
  3(a): Eyelets in supporting wear
  3(b): Inlet in supporting wear
  3(c): Front side of supporting wear
  3(d): Rear side of supporting wear
  4: Wearable attachment
  4(a): Straps of wearable attachment
  4(b): Attachment means of wearable attachment
  4(c): Inlet in wearable attachment
  4(d): Ropes for wearable attachment
  4(e): Holes in wearable attachment
  10: Fixed end of Sheath/Inlet of genital
  11: Sheath
  12: Sheath handle
  13: Protruding end of Sheath
  15: Isolation chamber
  16: Loop
  17: Straps
  18: Bound cover/Cap
100: Catheter
110: Air vent
120: Underwear Strap
121: Inlet for fluid
130: Scrotum means
131: Scrotum supporting wall
132: Drainage opening
133: Receiving opening for drainage
140(a): Proximate end of Isolation chamber
140(b): Distal end of Isolation chamber
140(c): Upper proximate body of Isolation chamber
140(d): Power proximate body of Isolation chamber
141: Fixing means of isolation chamber
150: Drainage Outlet
151: Inlet tube
156: Internal space
161: Spraying apparatus
170: Inward nozzle of Spraying apparatus
180: Outward nozzle of Spraying apparatus
181: Connector
190: Holding
192: Distal end of holding
193: Proximate end of holding
194: Upper portion of holding
195: Power portion of holding
200: Bidet
300: Urine collection device
```

We claim:

1. A male catheter system comprising a male catheter (100) and a support system (1) for the male catheter (100);
wherein,
said male catheter (100) comprises:
i. an isolation chamber (15) comprising a proximate end (140a), a distal end (140b), said proximate end (140a) further comprises an upper proximate body (140c) and a lower proximate body (140d), said upper proximate body (140c) having a plurality of fixing means (141);
ii. an inlet (10) for genital comprising a retractable non adhesive sheath (11) to receive and hold genital while in use;
iii. an inlet (121) for fluid;
iv. a spraying apparatus (161) comprising a channel for fluid and a plurality of nozzles (170, 180); and
v. a drainage outlet (150) comprising a drainage opening (132);
said support system (1) having a fore region (1a), an aft region (1b), a top region (1c) and a bottom region (1d), wherein said support system (1) includes:
i. a supporting wear (3) having a front side (3c) and a rear side (3d) with a plurality of eyelets (3a);
ii. a wearable attachment (4); and
iii. an optional bottom wear (2) having a plurality of eyelets (2a) and a flap (2b);
said male catheter (100) is attached on wearable attachment (4) at the fore region (1a) of said support system through a plurality of attachment means (4b); and
said wearable attachment (4) is attached to supporting wear (3).

2. The male catheter system as claimed in claim 1, wherein, the supporting wear (3) comprises of a plurality of eyelets (3a) and an inlet (3b) for genital.

3. The male catheter system as claimed in claim 1, wherein, the bottom wear (2) comprises of a plurality of eyelets (2a) and a flap (2b) act as a covering of the support system.

4. The male catheter system as claimed in claim 1, wherein, the wearable attachment (4) further comprises a plurality of tightening means and optionally a plurality of holes (4e) for channeling the tightening means.

5. The male catheter system as claimed in claim 4, wherein, the tightening means include but is not limited to a plurality of straps (4a), a plurality of ropes (4d).

6. The male catheter system as claimed in claim 5, wherein, the plurality of straps (4a) of the wearable attachment (4) are inserted through the plurality of eyelets (3a) on the front side (3c) of the supporting wear (3) and subsequently through the eyelets (2a) on front of the bottom wear (2) and secured on front of the bottom wear (2) and said plurality of straps (4a) traverse through rear side of the bottom wear (2) through the rear side (3d) of the supporting wear (3) and secured to rear side of said bottom wear (2) by a joining means.

7. The male catheter system as claimed in claim 5, wherein , the plurality of ropes (4d) are looped around the wearable attachment (4) and run around each thigh of user and come out from the plurality of eyelets (2a) on rear side of the bottom wear (2) through the plurality of eyelets (3a) on the rear side (3d) of the supporting wear (3).

8. The male catheter system as claimed in claim 5, wherein, the plurality of straps (4a) penetrates from the fore region (1a) of the support system (1) and exits from the aft region (1b) of the support system (1).

9. The male catheter system as claimed in claim 1, wherein, the fixing means (141) on the upper proximate body (140c) of the proximate end (140a) of the isolation chamber (15) is affixed to the attachment means (4b) of the wearable attachment (4), said wearable attachment (4) is attached to the supporting wear (3) and the bottom wear (2) through a plurality of tightening means thus completing the arrangement of the male catheter (100).

10. The male catheter system as claimed in claim 1, wherein, the fixing means (141) and attachment means (4b) include but not limited to buttons with eyelets, hooks, snap fasteners, press buttons, loop buttons.

11. The catheter system as claimed in claim 1, wherein the catheter is devoid of any adhesive.

12. A male catheter (100) comprising:
a) an isolation chamber (15) comprising a proximate end (140a) and a distal end (140b), said proximate end (140a) further comprises an upper proximate body (140c) and a lower proximate body (140d), said upper proximate body (140c) having a plurality of fixing means (141);
b) an inlet (10) for genital comprising a retractable non adhesive sheath (11) to receive and hold genital while in use;
c) an inlet (121) for fluid;
d) a spraying apparatus (161) comprising a channel for fluid and a plurality of nozzles (170, 180); and
e) a drainage outlet (150) comprising a drainage opening (132);
wherein;
the upper proximate body (140c) of said isolation chamber (15) is positioned such that said inlet (10) for genital is surrounded by said isolation chamber (15);
the inlet(121) for fluid is positioned preferably on upper proximate body (140c), said inlet (121) for fluid being connected to the spraying apparatus (161) forming a continued channel to direct a fluid to pass through the inlet (121) towards spraying apparatus (161) and to enter said isolation chamber (15);
the spraying apparatus (161) is positioned, inside and around the proximate end (140a) of the isolation chamber (15);
said drainage outlet (150) being positioned at distal end (140b) of said isolation chamber (15) such that the drainage opening (132) of said outlet (150) faces isolation chamber (15), and said drainage opening (132) protrudes allowing fluid collected in isolation chamber (15) to be drained out; and
the inlet (121) for fluid is optionally connected to a bidet (200) and said bidet provides pressurized fluid.

13. The catheter (100) as claimed in claim 12, wherein, the fluid includes water, air or douche.

14. The catheter (100) as claimed in claim 12, wherein, the bidet includes but is not limited to an automatic bidet, a manual bidet, a fluid container or similar structures known in the art.

15. The catheter (100) as claimed in claim 12, wherein, the upper proximate body (140c) of isolation chamber (15) is a soft flexible body, the lower proximate body (140d) is a semi-rigid body and the distal end (140b) of isolation chamber is a rigid body.

16. The catheter (100) as claimed in claim 12, wherein the catheter (100) collects fluid in the isolation chamber (15) allowing swift drainage of fluid thus avoiding contact of fluid with genital while in position.

17. The catheter (100) as claimed in claim 12, wherein, the catheter (100) allows for cleaning and/or washing of genital without removing the catheter (100) through spraying apparatus (161).

18. An external male catheter (100) comprising:
a) a holding (190) comprising a distal portion (192), a proximate portion (193), an upper portion (194) and a lower portion (195), a scrotum supporting means (130), a plurality of loop connectors (16) at said upper and said lower portions of said holding (190);
b) an isolation chamber (15) comprising a proximal end (140a) and a distal end (140b);
c) an inlet (10) for genital comprising a retractable, elastic, non-adhesive sheath (11) having a fixed end to receive the genital and a protruding end (13) to hold genital till upper half length of genital while in use;
d) an inlet (121) for fluid;
e) a spraying apparatus (161) comprising a channel for fluid and a plurality of nozzles (170,180); and
f) a drainage outlet (150) comprising a receiving opening (133) and a drainage opening (132);
wherein,
the inlet (10) for genital is positioned towards upper and proximate portions (194, 193) of said holding (190) such that said inlet for genital allows genital to be inserted into said inlet (10);
the proximal end (140a) of said isolation chamber (15) is connected to the upper and proximate portions (194, 193) of said holding such that said inlet (10) for genital is surrounded by said isolation chamber (15);
the inlet(121) for fluid is positioned preferably towards upper portion (194) of said holding, said inlet (121) for fluid being connected to the spraying apparatus (161) forming a continued channel to direct a fluid to pass through the inlet (121) towards spraying apparatus (161) and to enter into said isolation chamber (15);
the spraying apparatus (161) is positioned, inside and around the proximal end (140a) of the isolation chamber (15);
said drainage outlet (150) being positioned at distal portion (140b) of said isolation chamber (15) such that the receiving opening (133) of said outlet faces inner isolation chamber (15), and said drainage opening (132) protrudes out of said holding allowing fluid collected in isolation chamber (15) to be drained out; and
said inlet (121) for fluid is optionally connected to a bidet (200) and said bidet provides pressurized fluid.

19. The catheter (100) as claimed in claim 18, wherein, the fluid includes water, air or douche.

20. The catheter (100) as claimed in claim 18, wherein, the bidet includes but is not limited to an automatic bidet, a manual bidet, a fluid container or similar structures known in the art.
